(19) United States
(12) Patent Application Publication
(10) Pub. No.: US 2009/0005344 A1
(43) Pub. Date: Jan. 1, 2009

(54) Compounds and Methods of Use Thereof

(75) Inventors: James F. Burns, Raleigh, NC (US); Leonardo A. Cabana, Cary, NC (US); Glenn C. Collupy, Durham, NC (US); John R. Didsbury, Raleigh, NC (US); Tatyana A. Dyakonov, Durham, NC (US); Simon N. Haydar, Newton, PA (US); Michael L. Jones, Chapel Hill, NC (US); Francine F. Li, Chester Springs, PA (US); Christopher J. Markworth, Durham, NC (US); Jessymol Mathew, Raleigh, NC (US); Frank J. Schoenen, Lawrence, KS (US); David S. Van Vliet, Chapel Hill, NC (US); David Middlemiss, Bishop's Stortford (GB)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: NKUADA, LLC, Durham, NC (US)

(21) Appl. No.: 11/718,277

(22) PCT Filed: Oct. 27, 2005
(86) PCT No.: PCT/US05/38853

§ 371 Date: Apr. 17, 2008

Related U.S. Application Data
(60) Provisional application No. 60/624,057, filed on Nov. 1, 2004.

(51) Int. Cl. A61K 31/4184; A61K 31/381; A61P 43/00; A61P 29/00; A61P 19/02; A61P 17/06; A61P 19/08; A61P 11/06

(52) U.S. Cl. 514/64; 548/309.7; 514/394; 548/310.1; 548/181; 514/365; 548/110; 568/6; 548/304.4; 546/13

(57) ABSTRACT

Imidazole and benzimidazole boronic acid compounds, analogs thereof, and pharmaceutical formulations are described, along with methods of use thereof for inhibiting inflammatory cytokines such as tumor necrosis factor alpha (TNF-α) in a subject in need thereof.

### FIELD OF THE INVENTION

The present invention concerns benzimidazole boronic acid compounds, analogs thereof, pharmaceutical formulations containing the same, and methods of use thereof, particularly for inhibiting an inflammatory cytokine such as TNF-α in a subject in need thereof.

### BACKGROUND OF THE INVENTION

Tumor necrosis factor α (TNF-α) is an inflammatory cytokine produced by neutrophils, activated lymphocytes, macrophages, NK cells, LAK cells, astrocytes, and others. TNF-α mediates a variety of cellular activities, including cytotoxic effects against tumor cells, activation of neutrophils, growth proliferation of normal cells, and immunoinflammatory, immunoregulatory, and antiviral responses. Unfortunately TNF-α also mediates a variety of pathological activities in diverse number of disease states. See generally U.S. Pat. No. 5,643,893 to Benson et al.; see also PCT Application WO 00/73253 to Palladino et al. Accordingly there is a need for new inhibitors of TNF-α.

U.S. Pat. No. 5,643,893 to Benson et al. describes certain dihydroxyboryl alkyl purine, indole and pyrimidine derivatives that are useful as inhibitors of inflammatory cytokines. In general such inhibitors are compounds of the formula:

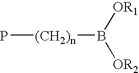

where R₁ and R₂ are both hydrogen atoms or together are a propylene chain bridging the two oxygen atoms; n is 2-6; and P is a purine, indole or pyrimidine base residue bonded via the N⁹ in the case of a purine base, or via the N¹ in the case of an indole or pyrimidine base. Certain specific substitutions, including 6- and 2,6-substituted purine derivatives, are also described.

PCT Application WO 02/085916 to Ishaq also describes certain dihydroxyboryl alkyl purine inhibitors of inflammatory cytokines of the formula:

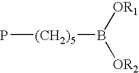

where P is a purine base, and R₁ and R₂ are both hydrogen atoms or together are a 3 to 5 carbon alkylene chain. Certain specific substitutions, including 6-, 2,6-, and 8-substituted purine derivatives, are also described (see, e.g., page 21 lines 6-7).

In spite of the foregoing there remains a need for new compounds for the inhibition of inflammatory cytokines such as TNF-α and methods of use thereof.

### SUMMARY OF THE INVENTION

A first aspect of the present invention is a compound of Formula I or Formula II:

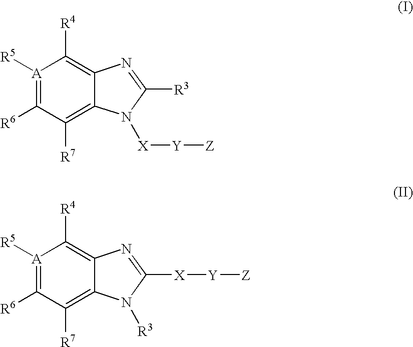

wherein:

A is N or C, subject to the proviso that R⁵ is absent when A is N;

X is —C(O)—, —S(O)₂—, or a covalent bond;

Y is linking group such as alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;

Z is selected from the group consisting of —B(OR)OR², —CON(R¹)OR², and —N(OR¹)COR² or any of the additional alternatives for Z described in greater detail below;

R¹ and R² are each independently H, loweralkyl, or together form C2-C4 alkylene; and

R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;

and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; and oxoheterocyclic groups;

or a pharmaceutically acceptable salt or prodrug thereof.

A father aspect of the invention is a method of inhibiting tumor necrosis factor alpha in a subject in need thereof, comprising administering a compound as described above to said subject in an amount effective to inhibit tumor necrosis factor alpha.

A further aspect of the invention is a method of inhibiting phosphodiesterase in a subject in need thereof, comprising administering a compound or active agent as described herein to the subject in an amount effective to inhibit phosphodiesterase (e.g., PDE II, PDE III, PDE IV, PDE V and combinations thereof such as both PDE II and PDE TV).

A further aspect of the invention is a method of treating an inflammatory disease in a subject in need thereof, comprising administering a compound or active agent as described herein to the subject in an amount effective to treat said inflammatory disease.

A further aspect of the invention is a method of treating inflammatory bowel disease in a subject in need thereof, comprising administering a compound or active agent as described herein to the subject in an amount effective to treat inflammatory bowel disease.

A further aspect of the invention is a method of treating rheumatoid arthritis in a subject in need thereof, comprising administering a compound or active agent as described herein to the subject in an amount effective to treat rheumatoid arthritis.

A further aspect of the invention is a method of treating psoriasis in a subject in need thereof, comprising administering a compound or active agent as described herein to the subject in an amount effective to treat psoriasis.

A further aspect of the invention is a method of treating ankylosing spondylitis in a subject in need thereof, comprising administering a compound or active agent as described herein to the subject in an amount effective to treat ankylosing spondylitis.

A further aspect of the invention is a method of treating psoriatic arthritis in a subject in need thereof, comprising administering a compound or active agent as described herein to the subject in an amount effective to treat psoriatic arthritis.

A further aspect of the invention is a method of treating asthma in a subject in need thereof, comprising administering a compound or active agent as described herein to the subject in an amount effective to treat asthma.

A further aspect of the invention is a method of treating chronic obstructive pulmonary disease in a subject in need thereof, comprising administering a compound or active agent as described herein to the subject in an amount effective to treat chronic obstructive pulmonary disease.

A further aspect of the invention is a method of treating Alzheimer's disease in a subject in need thereof, comprising administering a compound or active agent as described herein to the subject in an amount effective to treat Alzheimer's disease.

A further aspect of the invention is a method of treating type II diabetes in a subject in need thereof, comprising administering a compound or active agent as described herein to the subject in an amount effective to treat type II diabetes.

A further aspect of the invention is a method of treating cancer in a subject in need thereof, comprising administering a compound or active agent as described herein to the subject in an amount effective to treat cancer.

A further aspect of the invention is a method of treating hypertension in a subject in need thereof, comprising administering a compound or active agent as described herein to the subject in an amount effective to treat hypertension.

A further aspect of the invention is a method of treating erectile dysfunction in a subject in need thereof, comprising administering a compound or active agent as described herein to the subject in an amount effective to treat erectile dysfunction.

A further aspect of the invention is the use of a compound or active agent as described herein for the preparation of a medicament for carrying out a method as described herein.

The present invention is explained in greater detail below.

### DETAILED DESCRIPTION OF THE PREFERRED EMBODIMENTS

“Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Hydroxyl” as used herein refers to an —OH group.

“Nitro” as used herein refers to an —NO₂ group.

“Oxy” as used herein refers to a —O— group.

“Oxo” as used herein refers to a ═O group.

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3′-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. Alkyl and loweralkyl groups may be unsubstituted or substituted one or more times with halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkyl-amino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Alkenyl” as used herein alone or as part of another group, refers to a straight or branched chain hydrocarbon containing from 1 to 10 carbon atoms which include 1 to 4 double bonds in the normal chain. Representative examples of Alkenyl include, but are not limited to, vinyl, 2-propenyl, 3-butenyl, 2-butenyl, 4-pentyl, 3-pentyl, 2-hexenyl, 3-hexenyl, 2,4-heptadiene, and the like. These groups may be optionally substituted in like manner as described with alkyl above.

“Alkynyl” as used herein alone or as part of another group, refers to a straight or branched chain hydrocarbon containing from 1 to 10 carbon atoms which include 1 triple bond in the normal chain. Representative examples of Alkynyl include, but are not limited to, 2-propynyl, 3-butynyl, 2-butynyl, 4-pentenyl, 3-pentenyl, and the like These groups may be optionally substituted in like manner as described with alkyl above.

“Alkoxy,” as used herein alone or as part of another group, refers to am alkyl group, as defined herein, appended to the parent molecular moiety through an oxy group, as defined herein. Representative examples of alkoxy include, but are not limited to, methoxy, ethoxy, propoxy, 2-propoxy, butoxy, tert-butoxy, pentyloxy, hexyloxy and the like. These groups may be optionally substituted in like manner as described with alkyl above.

“Acyl” as used herein alone or as part of another group, refers to a —C(O)R radical, where R is any suitable substituent such as alkyl, alkenyl, alkynyl, aryl, alkylaryl, etc. as given herein.

“Haloalkyl,” as used herein alone or as part of another group, refers to at least one halogen, as defined herein, appended to the parent molecular moiety through am alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Alkylthio,” as used herein alone or as part of another group, refers to an alkyl group, as defined herein, appended to the parent molecular moiety through a thio moiety. Representative examples of alkylthio include, but are not limited, methylthio, ethylthio, tert-butylthio, hexylthio, and the like.

“Aryl,” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. These rings may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Arylalkyl,” as used herein alone or as part of another group, refers to an aryl group, as defined herein, appended to the parent molecular moiety through an alkyl group, as defined herein. Representative examples of arylalyl include, but are not limited to, benzyl, 2-phenylethyl, 3-phenylpropyl, 2-naphth-2-ylethyl, and the like.

“Amino” as used herein means the radical —NH₂.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, aryl, alkylaryl, etc.

“Amide” as used herein alone or as part of another group refers to a —C(O)NRₐR_{b} radical, where Rₐ and R_{b} are any suitable substituent such as alkyl, aryl, alkylaryl, etc.

“Sulfonamide” as used herein alone or as part of another group refers to a —S(O)₂NRₐR_{b} radical, where Rₐ and R_{b} are any suitable substituent, such as H, alkyl, aryl, alkylaryl, etc.

“Sulfone” as used herein alone or as part of another group refers to a —S(O)₂R radical, where R is any suitable substituent, such as H, alkyl, aryl, alkylaryl, etc.

“Aminosulfonyl” as used herein alone or as part of another group refers to a —N(Rₐ)S(O)₂R_{b} radical, where Rₐ and R_{b} are any suitable substituent, such as H, alkyl, aryl, alkylaryl, etc.

“Urea” as used herein alone or as part of another group refers to an —N(R_{c})C(O)NRₐR_{b} radical, where Rₐ, R_{b} and R_{c} are any suitable substituent such as H, alkyl, aryl, alkylaryl, etc.

“Alkoxyacylamino” as used herein alone or as part of another group refers to an —N(Rₐ)C(O)OR_{b} radical, where Rₐ, R_{b} are any suitable substituent such as H, alkyl, aryl, alkylaryl, etc.

“Aminoacyl” as used herein alone or as part of another group refers to an —C(O)NRₐR_{b} radical, where Rₐ and R_{b} are any suitable substituent, such as H, alkyl, aryl, alkylaryl, etc.

“Aminoacyloxy” as used herein alone or as part of another group refers to an —OC(O)NRₐR_{b} radical, where Rₐ and R_{b} are any suitable substituent, such as H, alkyl, aryl, alkylaryl, etc.

“Cycloalkyl,” as used herein alone or as part of another group, refers to a saturated or partially unsaturated cyclic hydrocarbon group containing from 3, 4 or 5 to 6, 7 or 8 carbons (which may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with halo or loweralkyl.

“Heterocyclic group” or “heterocycle” as used herein alone or as part of another group, refers to a monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1 or 2.

“Oxoheterocyclic group” refers to a heterocyclic group such as described above, substituted with one or more oxo groups, such as pyridine-N-oxide.

“Arylthio” as used herein refers to a group of the formula —S—R, where R is aryl as described above.

“Hydroxyamino” as used herein refers to a group of the formula —N(R)OH, where R is any suitable group such as alkyl, aryl, alkylaryl, etc.

“Treat” as used herein refers to any type of treatment that imparts a benefit to a patient afflicted with a disease, including improvement in the condition of the patient (e.g., in one or more symptoms), delay in the progression of the disease, etc.

“Inflammatory bowel disease.” as used herein includes both Crohn's disease and ulcerative colitis.

“Cancer” as used herein includes any cancer, particularly solid tumors, and includes but is not limited to lung cancer, colon cancer, breast cancer, prostate cancer, liver cancer, skin cancer, ovarian cancer, etc.

“Pharmaceutically acceptable” as used herein means that the compound or composition is suitable for administration to a subject to achieve the treatments described herein, without unduly deleterious side effects in light of the severity of the disease and necessity of the treatment.

“Pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299 Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

Prodrugs of the present invention include esters or compositions as described in U.S. Pat. No. 6,548,668 to Adams et al., U.S. Pat. No. 6,083,903 to Adams et al., or U.S. Pat. No. 6,699,835 to Plamondon et al., the disclosures of which are incorporated by reference herein in their entirety.

### 1. Active Compounds.

Active compounds of the present invention (this term including pharmaceutically acceptable salts and prodrugs thereof) can be made in accordance with known techniques (see, e.g., U.S. Pat. No. 5,643,893 to Benson et al.) or variations thereof which will be apparent to those skilled in the art based on the disclosure provided herein. In general active compounds of the invention are compounds of Formula I or Formula II:

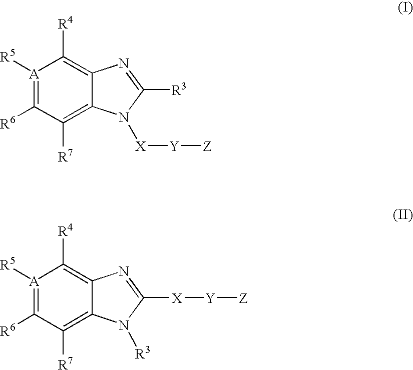

wherein:

A is N or C, subject to the proviso that R⁵ is absent when A is N;

X is, for Formula I, —C(O)—, —S(O)₂—, or a covalent bond, more preferably —S(O)₂—, or a covalent bond, and X is, for Formula II, —C(O)—, —S(O)₂—, or a covalent bond;

Y is a linking group such as alkyl (e.g., —R— where R is C2-C6 alkyl), alkenyl (e.g., —R— where R is C2-C6 alkenyl), cycloalkyl (e.g., —R— where R is C3-C6 cycloalkyl), alkylcycloalkyl(e.g., —R—R′—, where R is C1-C4 alkyl and R′ is C3-C6 cycloalkyl), cylcoalkylalkyl (e.g., —R—R′—, where R is C3-C6 cycloalkyl and R′ is C1-C4 alkyl), alkylcycloalkylalkyl (e.g., —R—R′—R″—, wherein R is C1-C4 alkyl, R′ is C3-C6 cycloalkyl, and R″ is C1-C4 alkyl), alkyloxyalkyl (e.g., —R—O—R′—, wherein R and R′ are C1-C4 alkyl); aryl (e.g., —R— where R is aryl), alkylaryl (e.g., —R—R′— where R is C1-C4 alkyl and R′ is aryl), alkylarylalkyl (e.g., —R—R′—R″— where R is C1-C4 alkyl, R′ is aryl, and R″ is C1-C4 alkyl), or arylalkyl (e.g., —R—R′— where R is aryl alkyl and R′ is C1-C4 alkyl); cycloalkylalkyl (e.g. —R—R′—, where R is C3-C6 cycloalkyl and R′ is C1-C4 alkyl), alkylheterocycle (e.g., —R—R′, where R is C1-C4 alkyl and R′ is a heterocyclic group as described herein), heterocyclealkyl, alkylheterocyclealkyl, heterocycle, aminoalkyl (e.g., —N(R)R′—, where R is H or C1-C4 alkyl and R′ is C1-C4 alkyl), oxyalkyl (e.g., —O—R— where R is C2-C6 alkyl), aminoaryl (e.g., —N(R)R′—, where R is H or C1-C4 alkyl and R′ is aryl), and oxyaryl (e.g., —O—R—, where R is aryl); and

Z is selected from the group consisting of —B(OR¹)OR², —CON(R¹)OR², and —N(OR¹)COR² or any of the additional alternatives for Z described in greater detail below.

R¹ and R² are each independently H, loweralkyl, or together form C2-C4 alkylene; and

R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio; and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;

or a pharmaceutically acceptable salt or prodrug thereof.

In some embodiments, R³ is preferably not H. Thus in some embodiments R³ is preferably a 5- or 6-membered organic ring containing 0 to 4 heteroatoms selected from the group consisting of N, O and S, which ring may be unsubstituted or substituted from 1 to 4 times with halo, cycloalkylalkoxy, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups.

It will be understood that, in Formula II where R³ is bonded to the ring nitrogen, it is less preferred for R³ to be halo, azido, mercapto, amino, alkylamino, dialkylamino, acylamino, cyano, and arylalkylamino, and more preferred for R³ to be alkyl, loweralkyl, and haloloweralkyl, sulfone, amide, and. aryl —

R⁵ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro. R⁵ is more preferably selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino. R⁵ is most preferably cyano, fluoroalkyl or halo.

R⁴ is in some embodiments preferably H. In other embodiments R⁴ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro; more preferably R⁴ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino, and still more preferably R⁴ is cyano, fluoroalkyl or halo.

In some embodiments R⁶ is H. In other embodiments R⁶ is preferably selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro, in such other embodiments R⁶ is more preferably selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino; in such other embodiments R⁶ is most preferably cyano, fluoroalkyl or halo.

In some embodiments, at least two of R⁴, R⁶, and R⁷ are H. In some preferred embodiments R⁶ and R⁷ are both H. In some preferred embodiments R⁷ is H.

Particularly preferred examples of compounds of the present invention are:
4-(2-(Trifluoromethyl)-1H-benzo[d]imidazol-1-yl)butylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;

and pharmaceutically acceptable salts and prodrugs thereof.

In addition, compounds of the present invention include compounds of Formula I and II above in which substituent -Z is a group of the formula:

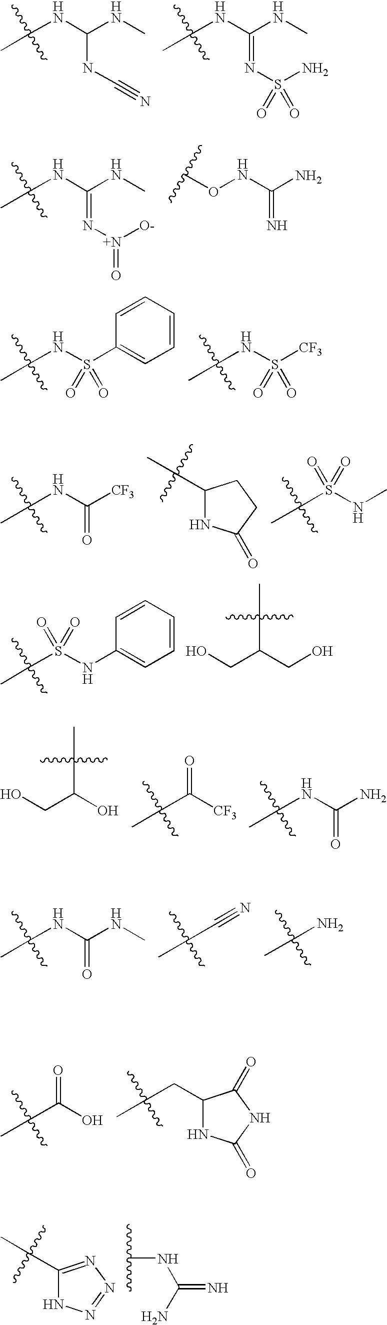

In addition, compounds of the present invention include compounds of Formula I and II above in which substituent —Y-Z is a group of the formula:

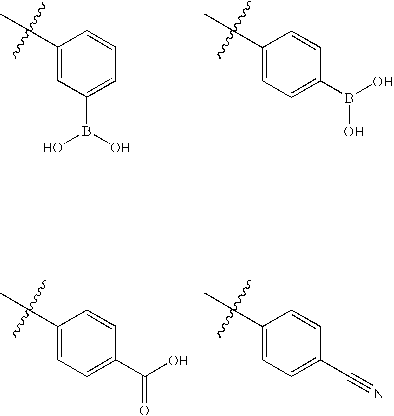

In addition, compounds of the invention include compounds of Formula I and II above in which the groups —X—Y-Z are a substituent of the formula:

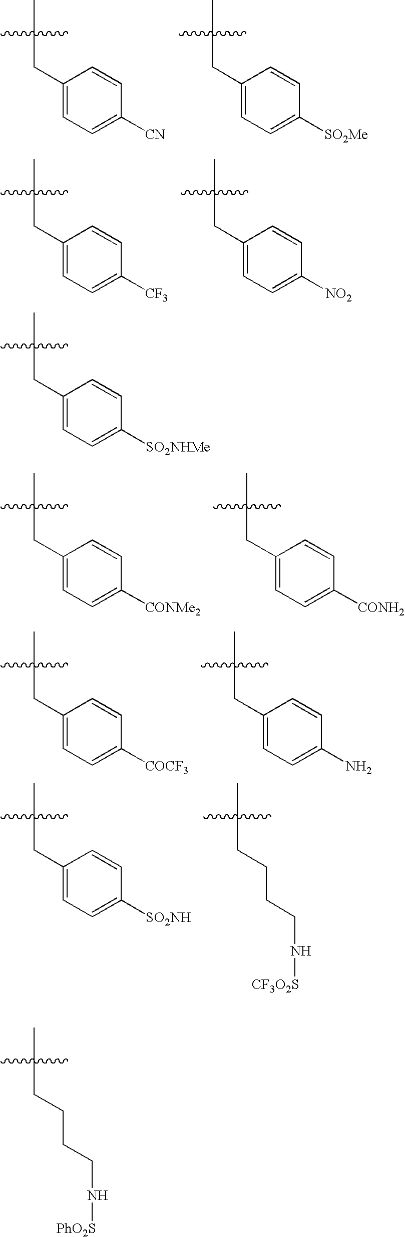

In addition, compounds of the invention include compounds of Formula I and II above in which the groups —X—Y-Z represent a substituent of the formula:

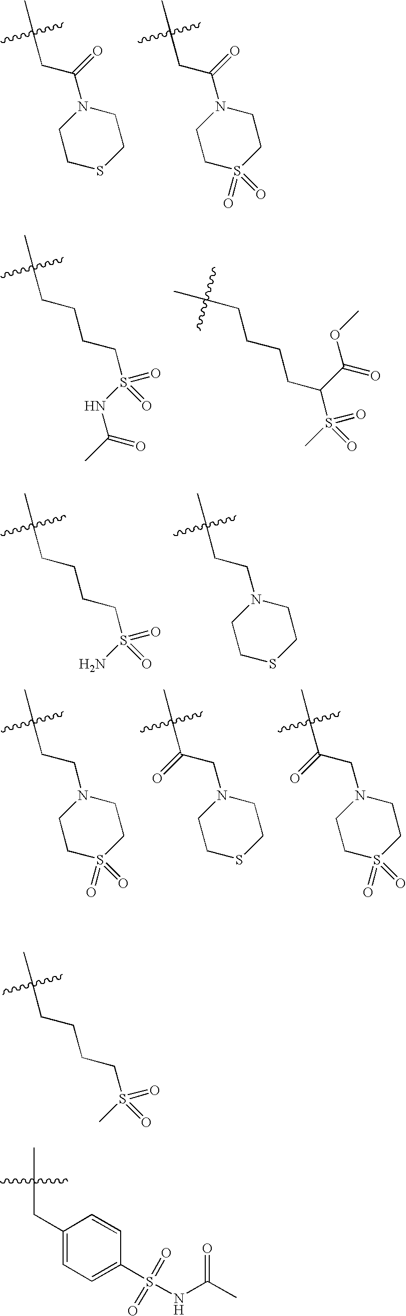

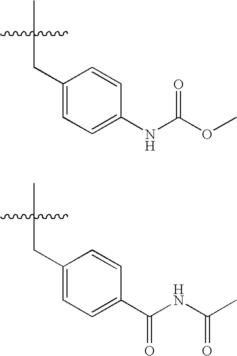

In addition, compounds of the invention include compounds of Formula I and II above in which group -Z is a substituent of the formula:

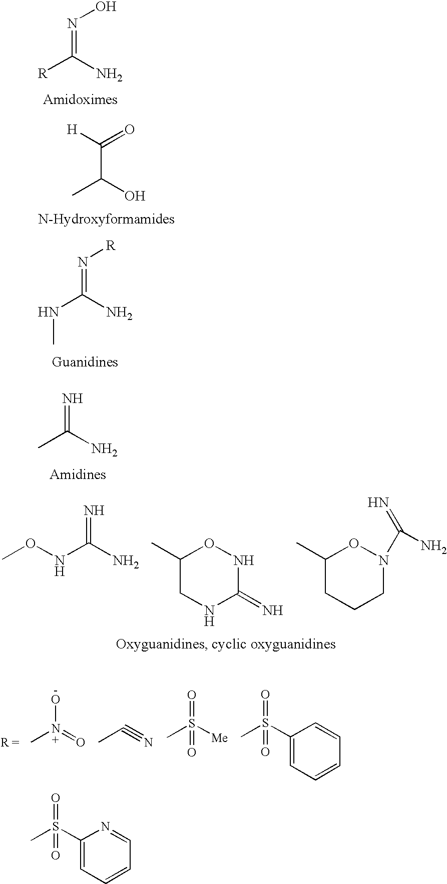

In addition, compounds of the invention includes compounds of the Formula I and II above in which group -Z is a substituent of the formula:

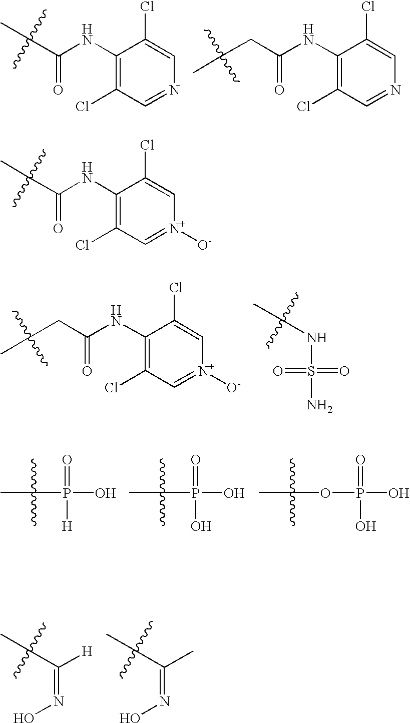

Examples of active compounds of the present invention include but are not limited to:

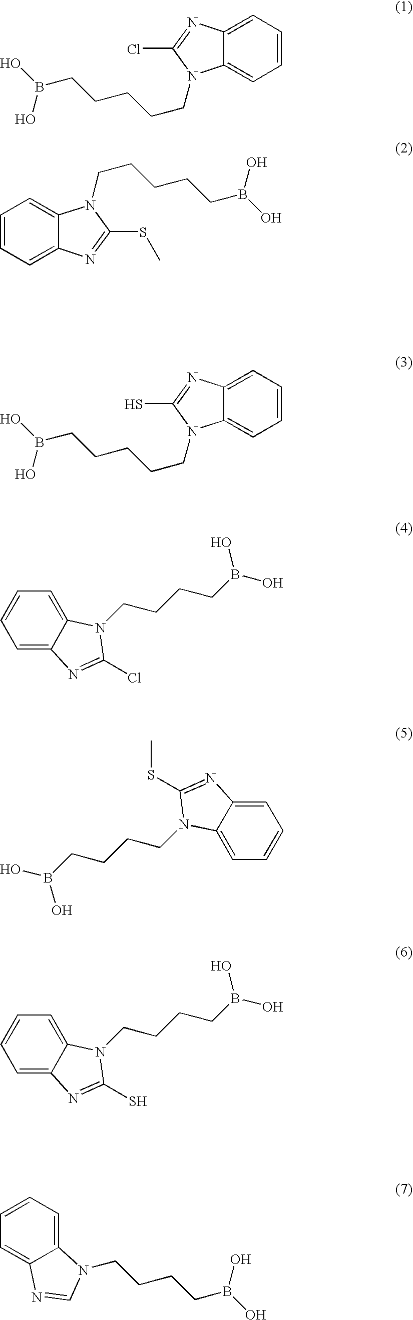

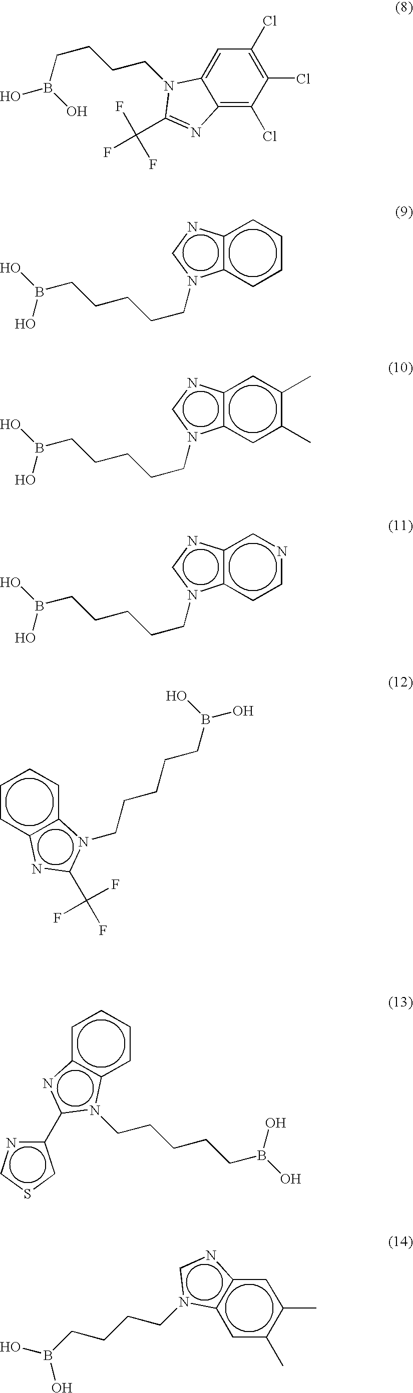

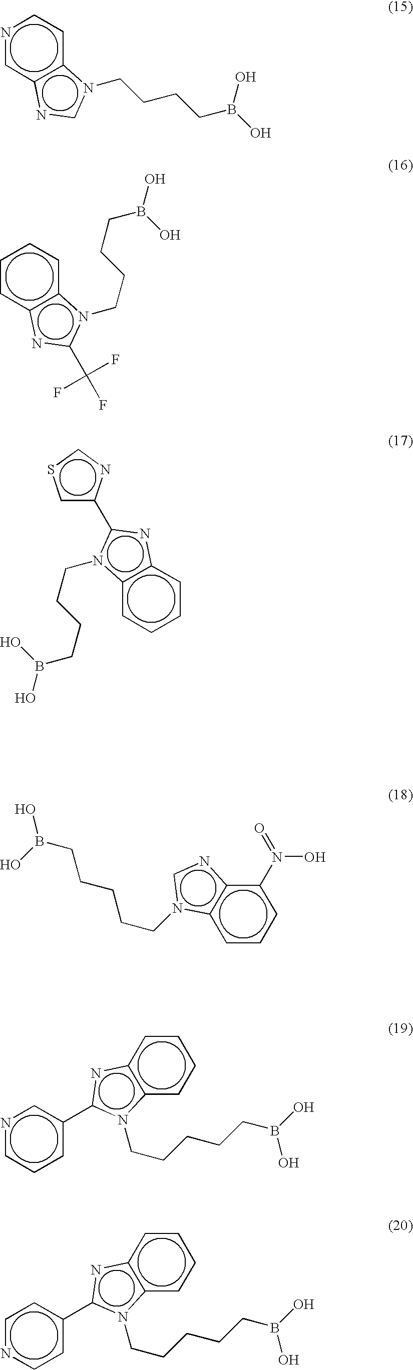

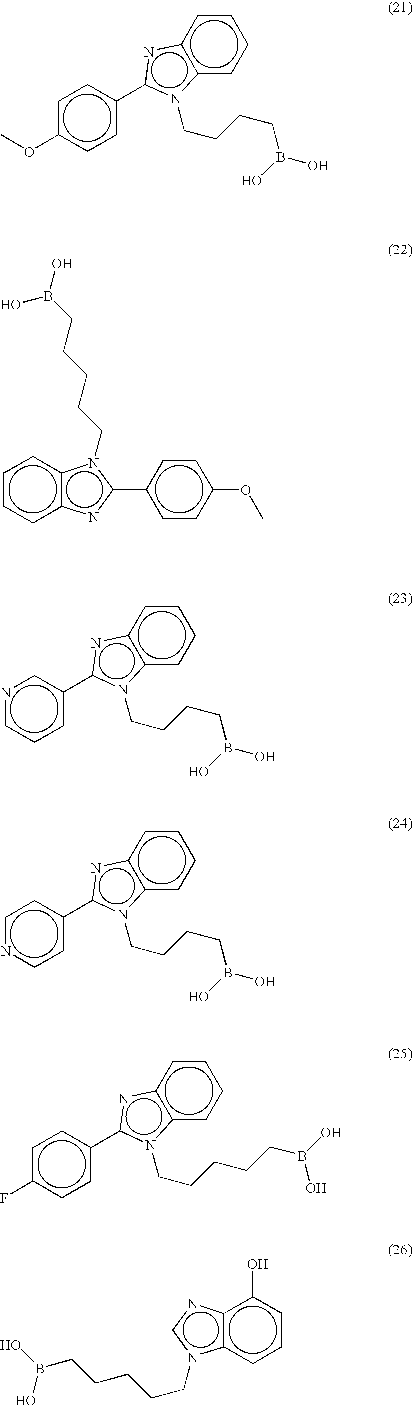

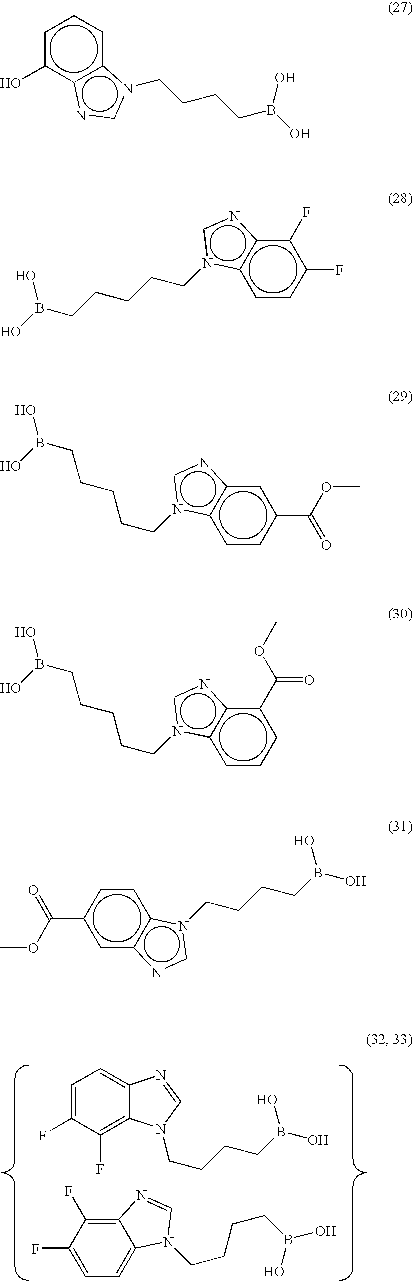

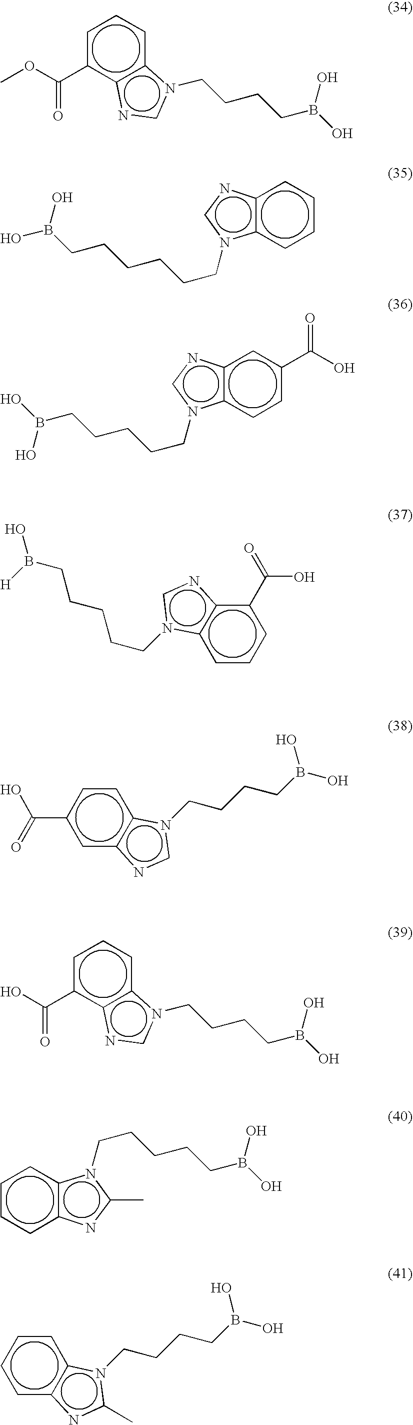

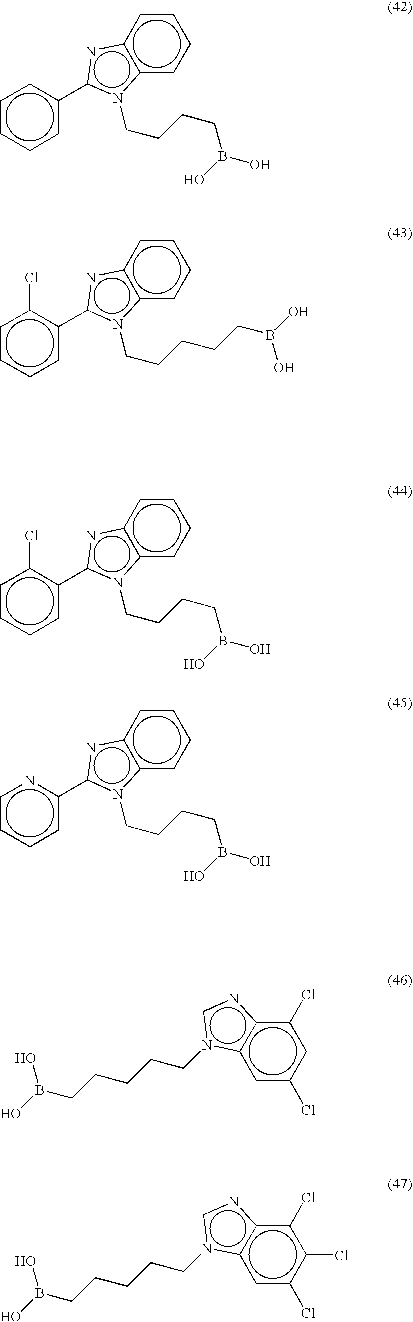

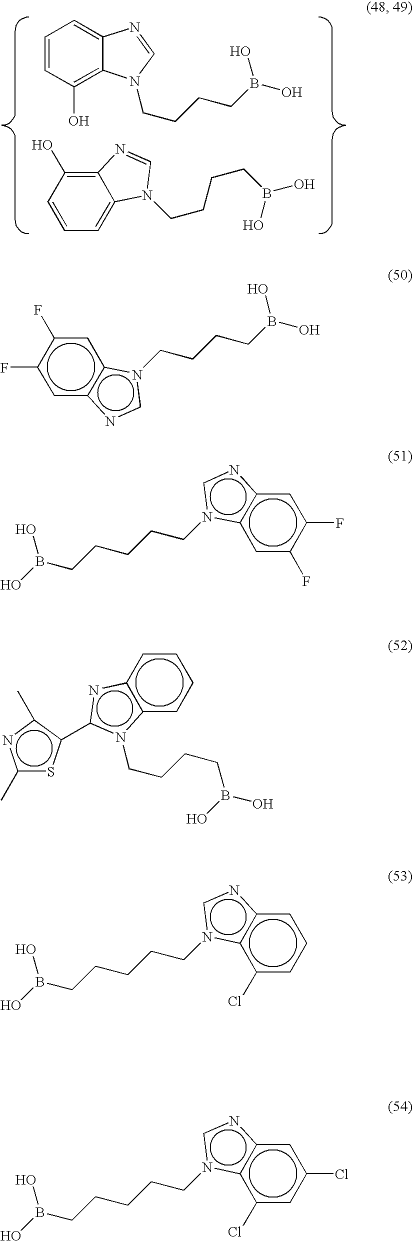

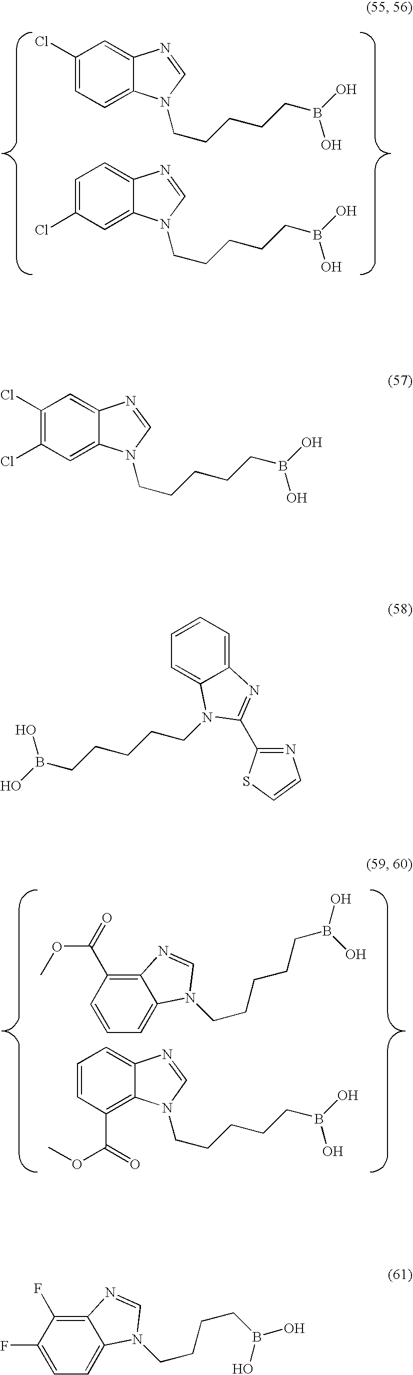

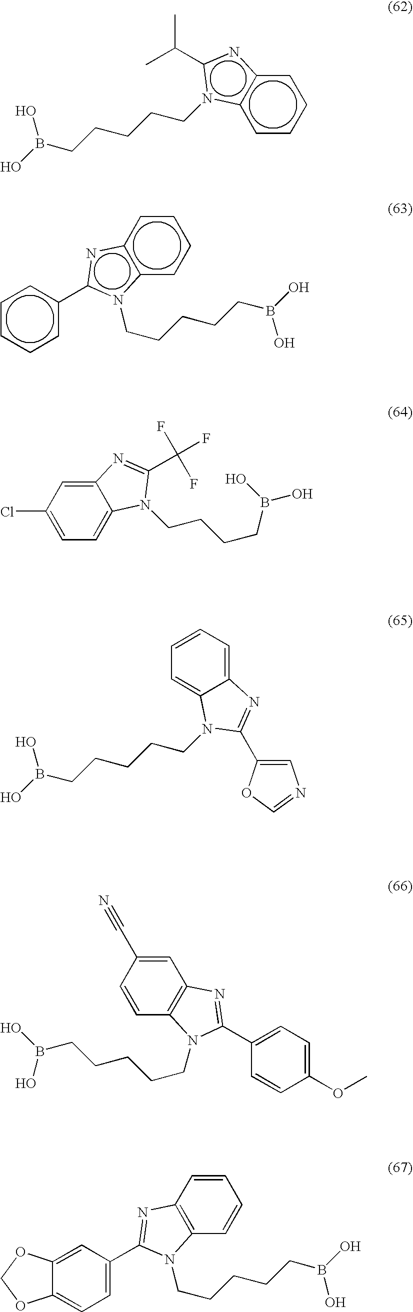

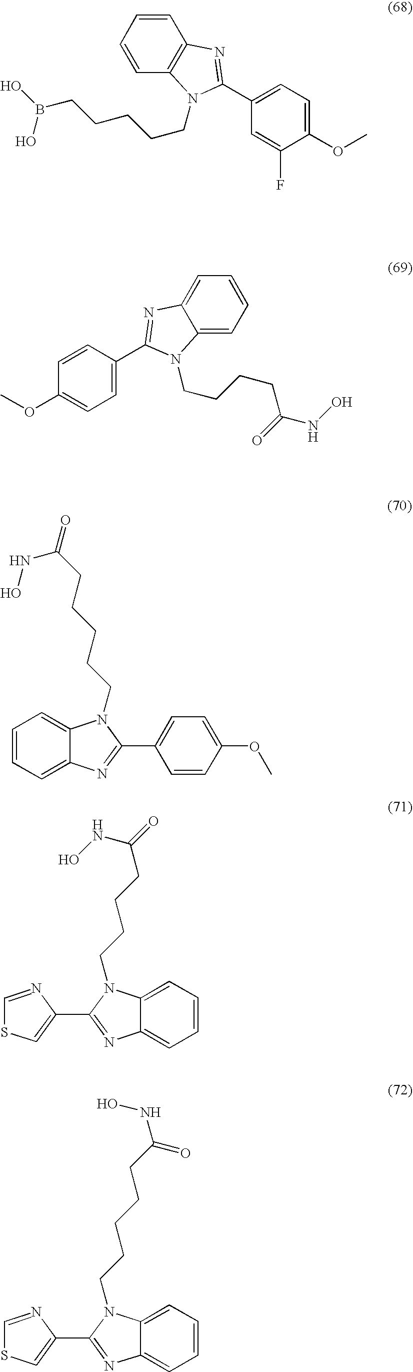

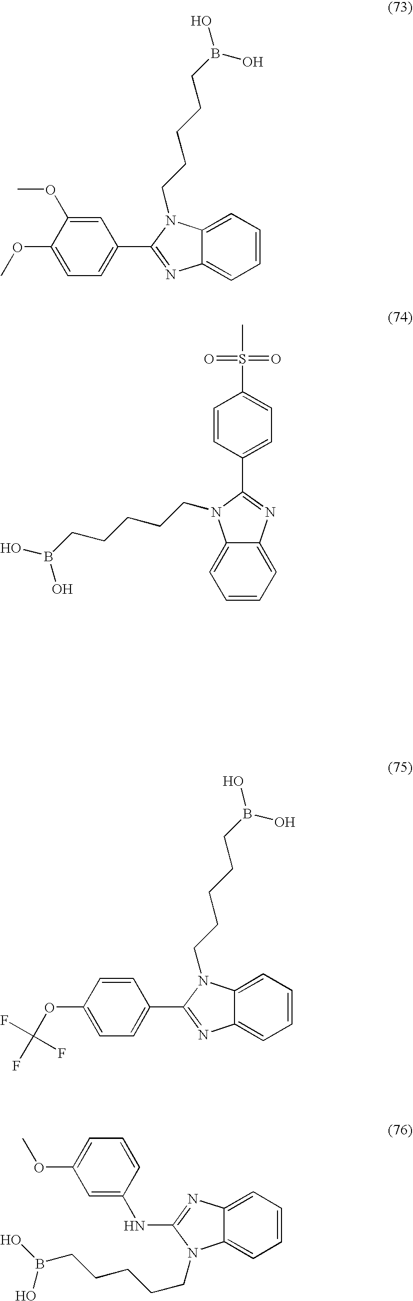

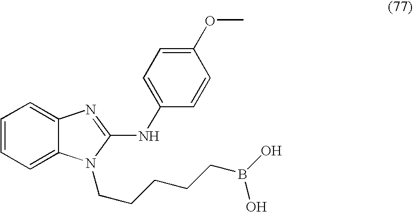

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

### 2. Pharmaceutical Formulations.

The active compounds described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavoured base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising a compound of Formula (I), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. Liposomal formulations containing the compounds disclosed herein or salts thereof, may be lyophilized to produce a lyophilizate which may be reconstituted with a pharmaceutically acceptable carrier, such as water, to regenerate a liposomal suspension.

Other pharmaceutical compositions may be prepared from the water-insoluble compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to the active compounds, the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. Of course, as indicated, the pharmaceutical compositions of the present invention may be lyophilized using techniques well known in the art.

### 3. Subjects.

The present invention is primarily concerned with the treatment of human subjects, but the invention may also be carried out on animal subjects, particularly mammalian subjects such as mice, rats, dogs, cats, livestock and horses for veterinary purposes, and for drug screening and drug development purposes.

Subjects to be treated with active compounds, or administered active compounds, of the present invention are, in general, subjects in which an inflammatory cytokine such as tumor necrosis factor alpha (TNF-α) is to be inhibited, and/or in which a phosphodiesterase (PDE) such as phosphodiesterase II, III, IV, and/or V is to be inhibited.

Subjects in need of treatment with active agents as described herein include, but are not limited to, subjects afflicted with invasive diseases, infections, and inflammatory diseases or states, such as: septic shock, cachexia (or weight loss associated with chronic diseases such as Alzheimer's disease, cancer, or AIDS), rheumatoid arthritis, inflammatory bowel disease (including but not limited to Crohn's disease and ulcerative colitis), multiple sclerosis, cogestive or chronic heart failure, psoriasis, asthma, non insulin-dependent diabetes mellitus, cerebral malaria, anemia associated with malaria, stroke, periodontitis, AIDS, and Alzheimer's disease. Subjects afflicted with such diseases are administered the active compound of the present invention (including salts thereof, alone or in combination with other compounds used to treat the said disease, in an amount effective to combat or treat the disease.

A particularly preferred category of diseases for treatment by the methods of the present invention are inflammatory diseases, or inflammations.

While it is presently believed that the aforesaid diseases are treated by the inhibitory effect of the active compounds described herein on TNF-α (and/or kinases implicated in inflammation), applicants do not wish to be bound to any specific theory of the invention, and it is intended that the treatment of particular diseases described herein by active compounds described herein be encompassed by the present invention without regard to the underlying physiological mechanism by which such treatment is accomplished.

### 4. Dosage and Routes of Administration.

As noted above, the present invention provides pharmaceutical formulations comprising the active compounds (including the pharmaceutically acceptable salts thereof), in pharmaceutically acceptable carriers for oral, rectal, topical, buccal, parenteral, intramuscular, intradermal, or intravenous, and transdermal administration.

The therapeutically effective dosage of any specific compound, the use of which is in the scope of present invention, will vary somewhat from compound to compound, and patient to patient, and will depend upon the condition of the patient and the route of delivery. In general, a dosage from about 0.05 or 0.1 to about 20, 50 or 100 mg/kg subject body weight may be utilized to carry out the present invention. For example, a dosage from about 0.1 mg/kg to about 50 or 100 mg/kg may be employed for oral administration; or a dosage of about 0.05 mg/kg to 20 or 50 mg/kg, or more, may be employed for intramuscular injection. The duration of the treatment may be one or two dosages per day for a period of two to three weeks, or until the condition is controlled or treated. In some embodiments lower doses given less frequently can be used prophylactically to prevent or reduce the incidence of recurrence of the condition being treated.

The present invention is explained in greater detail in the following non-limiting Examples.

### EXAMPLE 1

### 4-(2-(Trifluoromethyl)-1H-benzo[d]imidazol-1-yl)butylboronic acid

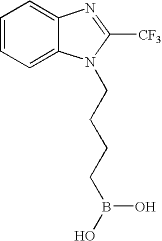

A 20 mL scintillation vial was charged with 2-(trifluoromethyl)benzimidazole (50 mg, 0.27 mmol, 1.0 equiv) and 95% sodium hydride (8 mg, 0.32 mmol, 1.2 equiv). Anhydrous dimethylformamide was added, and the reaction mixture was stirred for 10 min. A 1.0 M solution of 4-bromobutylboronic acid (53 mg, 0.30 nmol, 1.1 equiv) in dimethylformamide was added. The reaction was stirred at ambient temperature. After 5 days the reaction mixture was filtered through celite and concentrated in vacuo. The residue was purified by reverse-phase HPLC to afford 4-(2-(trifluoromethyl)-1H-benzo[d]imidazol-1-yl)butylboronic acid (43 mg, 53%): ¹H NMR (300 MHz, CD₃CN): δ 7.93 (d, J=8.0 Hz, 1H), 7.77 (d, J=8.0 Hz, 1H), 7.59 (t, J=7.4 Hz, 1H), 7.50 (m, 1H), 5.61 (s, 2H), 4.47 (t, f=7.7 Hz, 2H), 1.96 (pent, J=7.8 Hz, 2H), 1.57 (pent, J=7.8 Hz, 2H), 0.85 (t, J=7.9 Hz, 2H).

### EXAMPLES 2-4

### 5-(2-(Thiazol-4-yl)-1H-benzo[d]imidazol-1-yl)pentylboronic acid

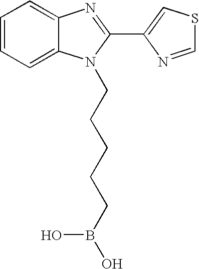

Cesium carbonate (486 mg, 1.50 mmol, 3.0 equiv) was added to a solution of thiabendazole (100 mg, 0.50 mmol, 1.0 equiv) in anhydrous dimethylformamide. After stirring for 10 min, a 1.0 M solution of 5-bromopentylboronic acid (145 mg, 0.75 μmmol, 1.5 equiv) was added. The reaction mixture was stirred at ambient temperature. After 5 h, the reaction mixture was filtered. Silica gel diol (1.1 g, 3 equiv) was added to the filtrate and shaken for 30 min. The silica gel was washed with 30 mL of acetonitrile followed by 30 mL of 95:5 water-acetonirile with 25 mmol trifluoroacetic acid. The aqueous wash was concentrated in vacuo, and the residue was purified by reverse-phase HPLC to afford 5-(2-(thiazol-4-yl)-1H-benzo[d]imidazol-1-yl)pentylboronic acid (110 mg, 70%).

A 1 dram vial was charged with thiabendazole (50 mg, 0.25 mmol, 1.0 equiv) and 95% sodium hydride (7.5 mg, 0.30 mmol, 1.2 equiv). Anhydrous dimethylformamide was added, and the reaction mixture was stirred for 10 min A 1.0 M solution of 5-bromopentylboronic acid (53 mg, 0.27 mmol, 1.1 equiv) in anhydrous dimethylformamide was added, and the reaction mixture was stirred at ambient temperature. After 4 days the reaction mixture was filtered and concentrated in vacuo. The residue was purified by reverse-phase HPLC to afford 5-(2-(thiazol-4-yl)-1H-benzo[d]imidazol-1-yl)pentylboronic acid (10.0 mg, 13%): ¹H NMR (300 MHz, CD₃CN): δ 9.39 (br s, 1H), 8.73 (br s, 1H), 7.88 (m, 1H), 7.72 (m, 1H), 7.46 (m, 2H), 4.72 (t, J=7.6 Hz, 2H), 1.71 (m, 2H), 1.21 (m, 2H), 0.43 (t, J=6.9 Hz, 2H).

Thiabendazole (10 g, 49.75 mmol) was added to a suspension of cesium carbonate (48.5 g, 149 mmol, 3.0 equiv) in dimethylformamide. After stirring for 30 min, a solution of bromopentylboronic acid (15 g, 77 mmol) was added. The reaction mixture was stirred for 2 days, then DI water was added until precipitate formed, product was filtered, then washed with water and filtered again. White solid was dried via vacuum (15 g, yield 96%). ¹H NMR (300 MHz, d6-DMSO): δ 9.39 (br s, 1H), 8.73 (br s, 1H), 7.88 (m, 1H), 7.72 (m, 1H), 7.46 (m, 2H), 4.72 (t, J=7.6 Hz, 2H), 1.71 (m, 2H), 1.21 (m, 2H), 0.43 (t, J=6.9 Hz, 2H). Elemental analysis: C, 56.99%, H, 5,91%, N, 13.33%.

### EXAMPLE 5

### 5-(5,6-dimethyl-1H-benzo[d]imidazol-1-yl)pentylboronic acid

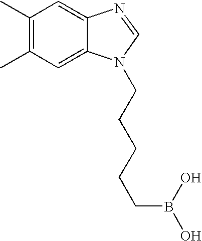

A suspension of 5,6-dimethylbenzimidazole (50 mg, 0.34 mmol) and potassium carbonate (70.9 mg, 0.51 mmol) in DMF (0.3 M) in a 40 mL scintillation vial was stirred for 30 min. A solution of 5-bromopentylboronic acid, (1 M, 0.0.38 mmol) was added and stirred at room temperature for 90 h. The reaction was filtered through celite and washed with DMF. The filtrate was evaporated and the residue was purified by HPLC to give 5-(5,6-dimethyl-1H-benzo[d]imidazol-1-yl)pentylboronic acid (12.4 mg, 14%). ¹H NMR (CD₃CN, 300 MHz) δ 8.794 (s, 1H), 7.65 (s, 1H), 7.585 (s, 1H), 4.333 (t, 2H, J=7.4 Hz), 2.425 (s, 3H), 2.398 (s, 3H), 1.444-1.269 (m, 4H), 0.66 (t, 21H, J=7.5 Hz)

### EXAMPLE 6

### 5-(1H-imidazo[4,5-c]pyridin-1-yl)pentylboronic acid

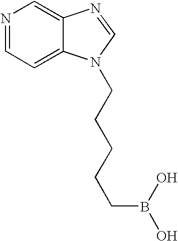

A suspension of 5-azabenzimidazole (50 mg, 0.42 mmol) and potassium carbonate (87.01 mg, 0.63 mmol) in DMF (0.3 M) in a 40 mL scintillation vial was stirred for 30 min. A solution of 5-bromopentylboronic acid, (1 M, 0.0.38 mmol) was added and stirred at room temperature for 90 h. The reaction was filtered through celite and washed with DMF. The filtrate was evaporated and the residue was purified by HPLC to give 5-(1H-imidazo[4,5-c]pyridin-1-yl)pentylboronic acid as a mixture of regioisomers (14.5 mg, 15%). ¹H NMR (CD₃CN) δ 9.25 (s), 9.194 (s), 8.622 (s, 1H), 8.549-8.487 (m, 1H), 8.106 (d, J=6 Hz), 8.035 (d, J=6.3 Hz), 4.553 (t, J=7.4), 4.385 (p, J=7.1 Hz), 1.963-1.871 (m, 2H), 1.456-1.267 (m, 4H), 0.694-0.631 (m, 2H).

### EXAMPLE 7

### 5-(2-(4-Methoxyphenyl)-1H-benzo[d]imidazol-1-yl)pentylboronic acid

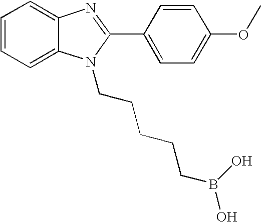

A 20 mL scintillation vial was charged with 2-(4-methoxyphenyl)-1H-benzo[d]imidazole (100 mg, 0.45 mmol, 1.0 eq), tetrabutylainmonium iodide (16 mg, 0.04 mmol, 0.1 eq), and 95% sodium hydride (26 mg, 1.04 mmol, 2.3 eq). Tetrahydrofuran was added to the vial, and the reaction mixture was stirred until gas evolution was no longer evident. A 1.0 M solution 5-bromopentylboronic acid (96 mg, 0.49 mmol, 1.5 eq) was added via syringe. The reaction mixture was stirred on a J-chem shaker at 180 rpm. After 48 h the reaction mixture was concentrated in vacuo. The residue was purified using an ISCO combiflash (12 g SiO₂, 30 ml/min, ethyl acetate to 9:1 ethyl acetate-methanol). The appropriate fractions were concentrated in vacuo and the resulting oil was lyophilized from 3:1 acetonitrile-water to afford 5-(2-(4-methoxyphenyl)-1H-benzo[d]imidazol-1-yl)pentylboronic acid (53 mg, 35%) as a white powder: ¹H NMR (400 MHz, d₆-DMSO): δ 7.67 (m, 2H), 7.60 (m, 1H), 7.36 (s, 2H), 7.22 (m, 1H), 7.10 (m, 1H), 7.10 (m, 2H), 4.22 (t, J=7.3 Hz, 2H), 3.82 (s, 3H), 1.64 (pent, J=7.4 Hz, 2H), 1.22 (pent, J=7.6 Hz, 2H), 1.09 (m, 2H), 0.46 (t, J=7.6 Hz, 2H).

### EXAMPLE 8

### 2-(3-Fluoro-4-methoxyphenyl)-1H-benzo[d]imidazole

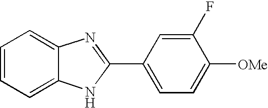

Samples of 3-fluoro-4-methoxybenzaldehyde (771 mg, 5 mmol) and 1,2-phenylenediamine (541 mg, 5 mmol) were suspended in nitrobenzene (2 mL) in a microwavable pressure tube (CEM). The mixture was subjected to microwave conditions (CEM Explorer, 200° C. and a hold time of 10 min). Upon cooling to room temperature, a large amount of a crystalline solid formed. The solid was filtered and triturated with hexane (3×20 mL) and hexane/EtOAc 4:1 (3×20 mL). The product was isolated as a tan solid (856 mg. 71%). ¹H NMR (400 MHz, CD₃CN): δ 7.84-7.89 (m, 2H), 7.60 (bs, 2H), 7.22-7.27 (m, 3H), 3.96 (s, 3H).

### EXAMPLE 9

### 2-(5-Bromopentyl)-4,4,5,5-tetramethyl-1,3,2-dioxaborolane

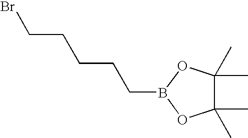

A solution of 5-bromopentylboronic acid (9.75 g, 50=mol) and pinacol (5.91 g, 50=mol) in acetonitrile (125 mL) was stirred at room temperature for 16 hr. The reaction mixture was concentrated under reduced pressure to give a dark gray residue. Purification using an Isco purification system (silica column, eluted with hexane/EtOAc 4:1) gave the product as a clear liquid (8.1 g, 58%). Visualization of the product in TLC analysis was achieved using anisaldehyde or KMnO₄ staining followed by heating. ¹H NMR (400 MHz, CD₃CN): δ 3.48 (t, J=6.8 Hz, 2H), 1.82-1.86 (m, 2H), 1.40-1.42 (m, 4H), 1.23 (s, 12H), 0.71-0.75 (m, 2H).

### EXAMPLE 10

### 2-(3-Fluoro-4-methoxyphenyl)-1-(5-(4,4,5,5-tetramethyl-1,3,2-dioxaborolan-2-yl)pentyl)-1H-benzo[d]imidazole

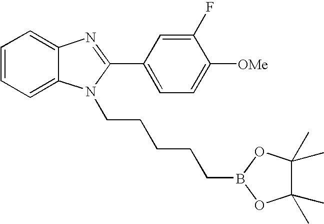

A suspension of 2-(3-fluoro-4-methoxyphenyl)-1-H-benzo[d]imidazole (300 mg, 1.24 mmol), 2-(5-bromopentyl)-4,4,5,5-tetramethyl-1,3,2-dioxaborolan (687 mg, 2.48 mmol) and cesium carbonate (808 mg, 2.48 mmol) in DMF (2.5 mL) was stirred at room temperature for 22 hr. The reaction mixture was diluted with EtOAc (25 mL) and H₂O (25 mL). The organic phase was extracted with aqueous LiCl (10%, 25 mL). The organic phase was dried (Na₂SO₄). The solvent was removed to afford a brown residue. Purification using an Isco purification system (silica column, eluted with hexane/EtOAc 4:1) gave the product as a clear liquid (8.1 g, 58%). ¹H NMR (400 MHz, CD₃CN): δ 7.53-7.68 (m, 1H), 7.50-7.52 (m, 3H), 7.24-7.30 (m, 3H), 4.24-4.28 (m, 2H), 3.96 (m, 3H), 1.71-1.75 (m, 2H), 1.10-1.30 (m, 16H), 0.58-0.62 (m, 2H).

### EXAMPLE 11

### 5-(2-(3-Fluoro-4-methoxyphenyl)-1H-benzo[d]imidazol-1-yl)pentylboronic acid

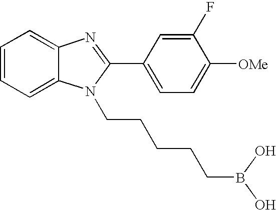

Samples of 2-(3-fluoro-4-methoxyphenyl)-1-(5-(4,4,5,5-tetramethyl-1,3,2-dioxaborolan-2-yl)pentyl)-1H-benzo[d]imidazole (810 mg, 1.85 mmol) and diethanolamine (2.1 g, 20 mmol) were combined in a microwavable pressure tube (CEM). The mixture was subjected to microwave conditions (CEM Explorer, 60° C. and a hold time of 10 min). LC-MS analysis showed some starting material. Another portion of diethanolamine (2.1 g, 20 mmol) was added to the viscous mixture. The mixture was again subjected to microwave conditions (60° C. and a hold time of 10 min). LC-MS analysis showed a trace of the starting material remaining. Thus, the reaction mixture was diluted with H₂O (50 mL) to form an emulsion. Extraction was performed sequentially using hexane (50 mL), hexane/EtOAc 4:1 (3×50 mL) and ether (2×50 mL). To the aqueous phase was added HCl (1M aqueous, 100 mL) followed by CH₂Cl₂ (100 mL). The mixture was stirred at room temperature for 20 min. The pH of the aqueous phase was adjusted to 8 using solid K₂CO₃. The organic phase was separated. The aqueous phase was extracted with CH₂Cl₂/EtOH 3:1 (3×100 mL). The organic phase was combined and dried (MgSO₄). The solvent was removed under reduced pressure to give an oily residue. Acetonitrile/H₂O 1:1 (20 mL) was added to the residue. After thorough mixing and solvent removal, an off-white solid was obtained. Trituration with hexane/EtOAc 4:1 (3×50 mL) afforded the material slightly contaminated with 2-(3-fluoro-4-methoxyphenyl)-1-(5-(4,4,5,5-tetramethyl-1,3,2-dioxaborolan-2-yl)pentyl)-1H-benzo[d]imidazole. The solid was then dissolved in acetone (5 mL) with heating. After cooling, the addition of hexane (30 mL) induced the precipitation of a white solid (250 mg, 38%). ¹H NMR (400 MHz, CD₃CN): δ 7.67-7.69 (m, 1H), 7.50-7.56 (m, 3)), 7.23-7.33 (m, 3H), 4.27 (t, J=8.0 Hz, 2H), 3.97 (s, 3H), 1.72-1.80 (m, 2H), 1.15-1.34 (m, 4H), 0.60 (t, J=8.0 Hz, 2H).

### EXAMPLE 12

### ethyl 6-(2-(thiazol-4-yl)-1H-benzo[d]imidazol-1-yl)hexanoate

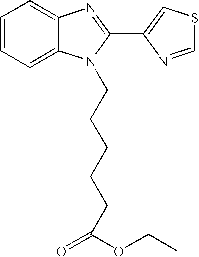

Cesium carbonate (2425 mg, 7.5 mmol, 3.0 equiv) was added to a solution of thiabendazole (500 mg, 2.48 mmol, 1.0 eqiv) in anhydrous dimethylformamide. After stirring for 30 min, a solution of ethyl 5-bromohexanoate (1106 mg, 4.96 mmol, 2 eqiv) was added. The reaction mixture was stirred for 3 hours. Then water (8:1) was added and this was extracted with ethyl acetate. The ethyl acetate solution was concentrated on vacuo and the residue was purified by silica gel column using ethyl acetate/hexane as an eluting solvent to afford ethyl 6-(2-(thiazol-4-yl)-1H-benzo[d]imidazol-1-yl)hexanoate. (650 mg, 76%): ¹H NMR (300 MHz, d6-DMSO): δ 9.32 (d, J=1.76 Hz, 1H), 8.48 (d, J=1.76 Hz, 1H), 7.64 (t,d, J=7.03 Hz, 1.7 Hz 2H), 7.25 (m, 2H), 4.72 (t, J=7.3 Hz, 2H, 3.99 (q, J=7.03 Hz, 2H), 2.19 (t, J=7.3 Hz, 2H), 1.73 (pent, J=7.3 Hz, 2H), 1.476 (pent, J=7.62 Hz, 2H), 1.23 (m, 2H), 1.106 (t, J=7.03 Hz, 3H).

### EXAMPLE 13

### N-hydroxy-6-(2-(thiazol-4-yl)-1H-benzo[d]imidazol-1-yl)hexanamide

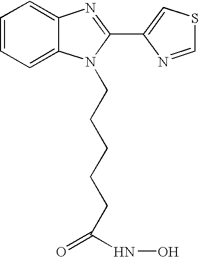

To a neat ethyl 6-(2-(thiazol-4-yl)-1H-benzo[d]imidazol-1-yl)hexanoate (400 mg, 1.16 mmol) N, O-Bis(trimethylsilyl)hydroxylamine (5,8 mmol, 1.03 g, 5 eq.) was added at room temperature. After stirring for 30 min a solution of 1N NaOH (2 ml) was added followed by the addition of methanol (˜7 ml). Then reaction mixture was concentrated via rotovap and then purified on silica gel column using methylene chloride/methanol as an eluting solvent (121 mg, 31%): ¹H NMR (300 MHz, d6-DMSO): δ 10.27 (s, 1H), 9.32 (d, J=2.345 Hz, 1H), 8.637 (s, 1H), 8.48 (d, J=1.759 Hz, 1H), 7.637 (t, J=8.793 Hz, 2H), 7.25 (m, 2H), 4.70 (t, J=7.33 Hz, 2H), 1.862 (t, J=7.33 Hz, 2H), 1.717 (t, J=7.33 Hz, 2H), 1.452 (t, J=7.33 Hz, 2H), 1.219 (m, 2H).

### EXAMPLE 14

### ethyl 5-(2-(thiazol-4-yl)-1H-benzo[d]imidazol-1-yl)pentanoate

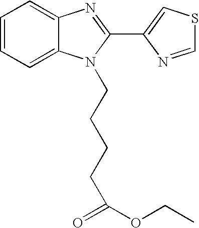

¹H NMR (300 MHz, d6-DMSO): δ 9.32 (d, J=1.759 Hz, 1H), 8.489 (d, J=2.345 Hz, 1H), 7.643 (t, J=6.741 Hz, 2H), 7.25 (m, 2H), 4.748 (t, J=7.034 Hz, 2H), 3.98 (q, J=7.6 Hz, 2H), 3.513 (t, J=6.448 Hz, 2H), 1.610 (pent, J=7.33 Hz, 2H), 1.477 (pent, J=7.622 Hz, 2H), 1.087 (t, J=7.034 Hz, 3H).

### EXAMPLE 15

### N-hydroxy-5-(2-(thiazol-4-yl)-1H-benzo[d]imidazol-1-yl)pentanamide

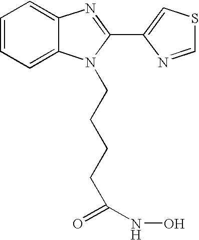

¹H NMR (300 MHz, d6-DMSO): δ 10.34 (broad, 1H), 9.438 (s, 1H), 8.754 (s, 1H), 7.88 (d, J=8.2 Hz, 1H), 7.76 (d, J=8.2 Hz, 1H), 7.47 (pent, J=5.5 Hz, 2H), 4.8 (t, J=7.034 Hz, 2H), 1.95 (t, J=7.3 Hz, 2H), 1.79 (pent, J=7.3 Hz, 2H), 1.52 (pent, J=7.62 Hz, 2H).

### EXAMPLE 16

### ethyl 5-(2-(4-methoxyphenyl)-1H-benzo[d]imidazol-1-yl)pentanoate

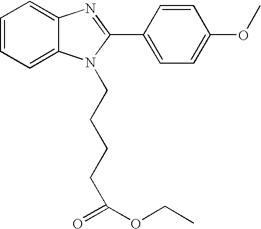

¹H NMR (300 MHz, d6-DMSO): δ 7.68 (d, J=8.79 Hz, 2H), 7.6 (m, 2H), 7.2 (m, 2H), 7.1 (d, J=8.79 Hz, 2H), 4.27 (t, J=7.3 Hz, 2H), 3.95 (q, J=7.034 Hz, 2H), 3.83 (s, 3H), 2.178 (t, J=7.3 Hz, 2H), 1.67 (m, 2H), 1.37 (pent, J=7.620 Hz, 2H), 1.096 (t, J=7.034 Hz, 3H).

### EXAMPLE 17

### N-hydroxy-5-(2-(4-methoxyphenyl)-1H-benzo[d]imidazol-1-yl)pentanamide

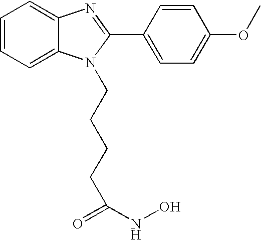

¹H NMR (300 MHz, d6-DMSO): δ 8.05 (d, J=7.62 Hz, 1H), 7.8 (d, J=8.79 Hz, 4H), 7,6(m, 2H), 7.25 (d, J=8.79 Hz, 2H), 4.43 (t, J=7.3 Hz, 2H), 3.88 (s, 3H) 1.88 (t, J=7.034 Hz, 2H), 1.74 (m, 2H), 1.44 (pent, J=7.62 Hz, 2H).

### EXAMPLE 18

### ethyl 6-(2-(4-methoxyphenyl)-1H-benzo[d]imidazol-1-yl)hexanoate

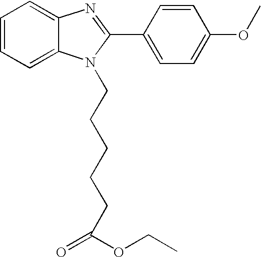

¹H NMR (300 MHz, d6-DMSO): δ 7.68 (d, J=8.79 Hz, 2H), 7.6 (m, 2H), 7.2 (m, 2H), 7.1 (d, J=8.79 Hz, 2H), 4.26 (q, J=7.3 Hz, 2H), 3.98 (m, 2H), 3.83 (s, 3H), 2.137 (t, J=7.3 Hz, 2H), 1.67 (m, 2H), 1.37 (m, 2H), 1.11 (m, 5H).

### EXAMPLE 19

### N-hydroxy-6-(2-(4-methoxyphenyl)-1H-benzo[d]imidazol-1-yl)hexanamide

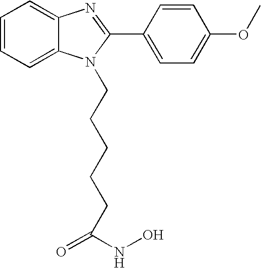

¹H NMR (300 MHz, d6-DMSO): δ 10.311 (broad, 1H), 7.856 (d, J=7.03 Hz, 1H), 7.76 (m, 3H), 7.433 (pent, J=5.8 Hz, 2H), 7.209 (d, J=8.79 Hz, 2H), 4.322 (t, J=7.3 Hz, 2H), 3.865 (s, 3H), 1.842 (t, J=7.3 Hz, 2H), 1.717 (pent, J=7.034 Hz, 2H), 1.385 (pent, J=7.3 Hz, 2H), 1.147 (m, 2H).

### EXAMPLE 20

### Bilogical Example

### Inhibition of TNF-α Production By Peripheral Blood Monocyte Cells (PMBC)

PMBC in RPMI 1640 Cell Culture Medium (containing 1% Penicillin and 1% Streptomycin) are aliquoted into 96-well plates at 5×10⁵ cells/well and pre-incubated with test compounds for 30 minutes at 37° C. After incubation, 1 ug/mL LPS is added to each well to stimulate TNF-α production and the plate is incubated for 24 hours at 37° C. After incubation, the supernatant is removed and the TNF-α secreted is quantified using EIA detection kits commercially available from R&D Systems (USA). The results from this assay are expressed as percent inhibition of control activity, with the control being stimulated wells with no test compound. Dexamethasone is used as a standard reference compound in the assay and is tested with each experiment. All test compounds are diluted from 10 mM stock solutions in 100% DMSO.

TABLE 1
TNF-α IC50 Values

| Compound Example | |
|---|---|
| Number | IC₅₀ |
| (1) | — |
| (2) | 560 nM |
| (3) | — |
| (4) | — |
| (5) | — |
| (6) | — |
| (7) | 300 nM |
| (8) | — |
| (9) | — |
| (10) | — |
| (11) | 500 nM |

The foregoing is illustrative of the present invention, and is not to be construed as limiting thereof. The invention is defined by the following claims, with equivalents of the claims to be included therein

That which is claimed is:

1. A compound of Formula I or Formula II: 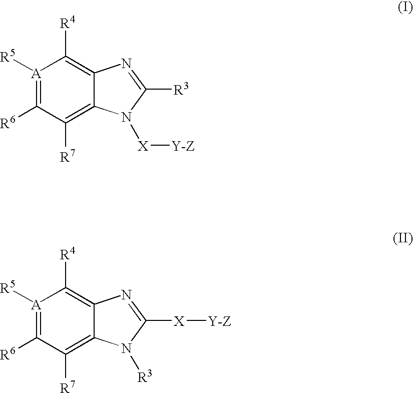
wherein:
A is N or C, subject to the proviso that R⁵ is absent when A is N;
X is —C(O)—, —S(O)₂—, or a covalent bond;
Y is alkyl, alkenyl, cycloalkyl, alkylcycloalkyl, alkylcycloalkylalkyl, alkyloxyalkyl, aryl, alkylaryl, alkylarylalkyl, arylalkyl, cycloalkylalkyl, alkylheterocycle, heterocyclealkyl, alkylheterocyclealkyl, heterocycle, aminoalkyl, oxyalkyl, aminoaryl, oxyaryl;
Z is selected from the group consisting of —B(OR¹)OR², —CON(R¹)OR², and —N(OR¹)COR²;
R¹ and R² are each independently H, loweralkyl, or together form C2-C4 alkylene; and
R³, R⁴, R⁵, R⁶, and R⁷ are each independently selected from the group consisting of: H, halo, loweralkyl, haloloweralkyl, haloloweralkoxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; arylalkyloxy, cycloalkyloxy, cycloalkylalkoxy, cycloalkylamino, urea, cycloalkylalkylamino, cycloalkyl, alkylcycloalkyl, hydroxyamino, alkoxyacylamino, and arylthio;
and 5- or 6-membered organic rings containing 0 to 4 heteroatoms selected from the group consisting of N, O and S, which rings may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups;
or a pharmaceutically acceptable salt or prodrug thereof.

2. The compound of claim 1, wherein R⁵ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro, and hydroxyamino.

3. The compound of claim 1, wherein R⁵ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino.

4. The compound of claim 1, wherein R⁵ is cyano, fluoroalkyl or halo.

5. The compound of claim 1, wherein R⁴ is H.

6. The compound of claim 1, wherein R⁴ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro.

7. The compound of claim 1, wherein R⁴ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino.

8. The compound of claim 1, wherein R⁴ is cyano, fluoroalkyl or halo.

9. The compound of claim 1, wherein R⁶ is H.

10. The compound of claim 1, wherein R⁶ is selected from the group consisting of: halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, and nitro.

11. The compound of claim 1, wherein R⁶ is selected from the group consisting of: halo, haloloweralkyl, haloloweralkyloxy, loweralkoxy, amino, acylamino, aminoacyl, arylalkyl, aryloxy, acyl, arylamino, cyano, nitro, and heterocycleamino.

12. The compound of claim 1, wherein R⁶ is cyano, fluoroalkyl or halo.

13. The compound of claim 1, wherein R⁷ is H.

14. The compound of claim 1, wherein at least two of R⁴, R⁶, and R⁷ are H.

15. The compound of claim 1, wherein R⁶ and R⁷ are H.

16. The compound of claim 1, wherein A is N.

17. The compound of claim 1, wherein A is C.

18. The compound of claim 1, wherein R³ is a 5- or 6-membered organic ring containing 0 to 4 heteroatoms selected from the group consisting of N, O and S, which ring may be unsubstituted or substituted from 1 to 4 times with halo, loweralkyl, haloloweralkyl, haloloweralkyloxy, loweralkoxy, hydroxy, loweralkoxycarbo, carboxylic acid, acyl, azido, mercapto, alkylthio, amino, heterocycleamino, alkylamino, dialkylamino, acylamino, aminoacyl, arylamino, arylalkyl, arylalkylamino, aryloxy, cyano, sulfonamide, aminosulfonyl, sulfone, nitro; and oxoheterocyclic groups.

19. The compound of claim 1, wherein said compound is selected from the group consisting of:
4-(2-(Trifluoromethyl)-1H-benzo[d]imidazol-1-ylbutylboronic acid;
5-(2-(Thiazol-4-yl)-1H-benzo[d]imidazol-1-yl)pentylboronic acid;
5-(5,6-dimethyl-1H-benzo[d]imidazol-1-yl)pentylboronic acid;
5-(1H-imidazo[4,5-c]pyridin-1-yl)pentylboronic acid;
5-(2-(4-Methoxyphenyl)-1H-benzo[d]imidazol-1-yl)pentylboronic acid;
5-(2-(3-Fluoro-4-methoxyphenyl)-1H-benzo[d]imidazol-1-yl)pentylboronic acid;
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

20. The compound of claim 1, wherein said compound is 5-(2-(Thiazol-4-yl)-1H-benzo[d]imidazol-1-yl)pentylboronic acid and pharmaceutically acceptable salts and prodrugs thereof.

21. The compound of claim 1, wherein said compound is 5-(2-(4-Methoxyphenyl)-1H-benzo[d]imidazol-1-yl)pentylboronic acid and pharmaceutically acceptable salts and prodrugs thereof.

22. The compound of claim 1, wherein said compound is 5-(2-(3-Fluoro-4-methoxyphenyl)-1H-benzo[d]imidazol-1-yl)pentylboronic acid and pharmaceutically acceptable salts and prodrugs thereof.

23. The compound of claim 1, wherein said compound is selected from the group consisting of:
5-(5-cyano-2-(4-methoxyphenyl)-1H-benzo[d]imidazol-1-yl)pentylboronic acid;
5-(6-cyano-2-(4-methoxyphenyl)-1H-benzo[d]imidazol-1-yl)pentylboronic acid;
and pharmaceutically acceptable salts and prodrugs thereof.

24. A pharmaceutical composition comprising a compound of claim 1 in a pharmaceutically acceptable carrier.

25. The composition of claim 24, wherein said carrier is an aqueous carrier.

26. A method of inhibiting tumor necrosis factor alpha in a subject in need thereof, comprising administering a compound of claim 1 to said subject in an amount effective to inhibit tumor necrosis factor alpha.

27. A method of inhibiting phosphodiesterase in a subject in need thereof, comprising administering a compound of claim 1 to said subject in an amount effective to inhibit phosphodiesterase.

28. A method of claim 27, wherein said phosphodiesterase (PDE) is selected from the group consisting of PDE II, PDE III, PDE IV, PDE V and combinations thereof.

29. A method of treating an inflammatory disease in a subject in need thereof, comprising administering a compound of claim 1 to said subject in an amount effective to treat said inflammatory disease.

30. A method of treating inflammatory bowel disease in a subject in need thereof, comprising administering a compound of claim 1 to said subject in an amount effective to treat inflammatory bowel disease.

31. A method of treating rheumatoid arthritis in a subject in need thereof, comprising administering a compound of claim 1 to said subject in an amount effective to treat rheumatoid arthritis.

32. A method of treating psoriasis in a subject in need thereof, comprising administering a compound of claim 1 to said subject in an amount effective to treat psoriasis.

33. A method of treating ankylosing spondylitis in a subject in need thereof, comprising administering a compound of claim 1 to said subject in an amount effective to treat ankylosing spondylitis.

34. A method of treating psoriatic arthritis in a subject in need thereof, comprising administering a compound of claim 1 to said subject in an amount effective to treat psoriatic arthritis.

35. A method of treating asthma in a subject in need thereof, comprising administering a compound of claim 1 to said subject in an amount effective to treat asthma.

36. A method of treating chronic obstructive pulmonary disease in a subject in need thereof, comprising administering a compound of claim 1 to said subject in an amount effective to treat chronic obstructive pulmonary disease.

37. A method of treating Alzheimer's disease in a subject in need thereof, comprising administering a compound of claim 1 to said subject in an amount effective to treat Alzheimer's disease.

38. A method of treating type II diabetes in a subject in need thereof, comprising administering a compound of claim 1 to said subject in an amount effective to treat type II diabetes.

39. A method of treating cancer in a subject in need thereof, comprising administering a compound of claim 1 to said subject in an amount effective to treat cancer.

40. A method of treating hypertension in a subject in need thereof, comprising administering a compound of claim 1 to said subject in an amount effective to treat hypertension.

41. A method of treating erectile dysfunction in a subject in need thereof, comprising administering a compound of claim 1 to said subject in an amount effective to treat erectile dysfunction.
